(19) United States
(12) Patent Application Publication
(10) Pub. No.: US 2024/0252735 A1
(43) Pub. Date: Aug. 1, 2024

(54) MEDICAL SUCTION PUMP AND MEDICAL SUCTION DEVICE INCLUDING THE SAME

(71) Applicant: LHBioMed Co., Ltd., Wonju-si (KR)

(72) Inventor: Seong Soo HONG, Yongin-si (KR)

(73) Assignee: LHBioMed Co., Ltd., Wonju-si (KR)

(21) Appl. No.: 18/429,803

(22) Filed: Feb. 1, 2024

(30) Foreign Application Priority Data
Feb. 1, 2023 (KR) 10-2023-0013812

(51) Int. Cl. A61M 1/00

(52) U.S. Cl. CPC A61M 1/81; A61M 1/74

(57) ABSTRACT

A medical suction pump according to one embodiment of the present disclosure includes: a body; a motor provided in the body; a rotational shaft connected to the motor and configured to rotate according to a driving of the motor; a crank having one end connected to the rotational shaft and configured to convert a rotational motion of the rotational shaft into a linear reciprocating motion; a piston including a connecting rod connected to the other end of the crank and a piston head connected to the connecting rod; and a cylinder configured to accommodate the piston head therein and having a suction/exhaust port formed therein. The suction/exhaust port of the cylinder is connected to a storage in which objects suctioned via a tube are stored, and a negative pressure is generated when the piston head accommodated in the cylinder moves in a first direction and a second direction opposite to the first direction.

### CROSS-REFERENCE TO RELATED APPLICATION

This application claims priority to Korean Patent Application No. 10-2023-0013812 filed on Feb. 1, 2023, the entire contents of which are herein incorporated by reference.

### TECHNICAL FIELD

The present disclosure relates to a medical suction pump, and more particularly to a suction pump used in a medical suction device that suctions objects to be suctioned or contaminated water during a medical procedure.

### BACKGROUND

In general, when treating a patient at a hospital, a suction device is used to perform a suction treatment of suctioning objects to be suctioned and contaminated water at a treatment site. In the related art, a hydraulic pump may be used for the suction treatment. That is, an operator grips a hand piece and uses the hydraulic pump to generate a negative pressure in the hand piece. Thus, through the hand piece, the operator may perform the suction treatment of suctioning objects to be suctioned or contaminated water at a treatment site.

As an example, the hydraulic pump may be used for a suction treatment such as a liposuction procedure among plastic surgical procedures.

The liposuction is a kind of a cosmetic surgical procedure which suctions and removes a fat layer beneath the accumulated skin for the purpose of diet and body contouring. As a representative liposuction, a suction-assisted lipoplasty (SAL), which is a method of suctioning and removing fat with a cannula by using a negative pressure generated by a suction pump, is used.

A liposuction device in the related art uses a hydraulic pump to suction and remove crushed and emulsified fat tissues. However, such a hydraulic pump generates a relatively large operational noise of 75 decibel (dB) or more, which may cause stress to the operator and the patient during the procedure. In addition, the size and weight of the hydraulic pump may cause a degradation of mobility. For this reason, the hydraulic pump is mainly installed outdoors and performs the suction treatment using a connection hose. However, the installation of the hydraulic pump outdoors may result in an increase in cost, and the hydraulic pump may operate abnormally due to an outdoor temperature.

Accordingly, there is a demand for the development of a device capable of performing the suction treatment in a convenient and efficient manner while minimizing the generation of stress due to the operational noise.

### SUMMARY

One object of the present disclosure is to solve the above-described problems in the prior art.

Another object of the present disclosure is to provide a medical suction device capable of reducing noise during an operation of a medical suction pump.

Yet another object of the present disclosure is to provide a medical suction device having an improved mobility by reducing a size and weight of the medical suction device.

Representative configurations of the present disclosure to achieve the above objects are described below.

According to one aspect of the present disclosure, there is provided a medical suction pump includes: a body; a motor provided in the body; a rotational shaft connected to the motor and configured to rotate according to a driving of the motor; a crank having one end connected to the rotational shaft and configured to convert a rotational motion of the rotational shaft into a linear reciprocating motion; a piston including a connecting rod connected to the other end of the crank and a piston head connected to the connecting rod; and a cylinder configured to accommodate the piston head therein and having a suction/exhaust port formed therein. The suction/exhaust port of the cylinder is configured to be connected to a storage in which objects suctioned via a tube are stored, and a negative pressure is generated when the piston head accommodated in the cylinder moves in a first direction and a second direction opposite to the first direction.

According to one aspect of the present disclosure, the cylinder, the piston, and the crank may include a pair of cylinders, a pair of pistons and a pair of cranks, respectively, which are provided on both sides of the body.

According to one aspect of the present disclosure, the pair of cranks may be configured such that the one ends connected to the rotational shaft faces each other, and reciprocating motions of the pair of pistons respectively connected to the pair of cranks may have opposite phases.

According to one aspect of the present disclosure, the suction/exhaust port formed in each of the pair of cylinders may be connected to a medical suction device via a T-shaped connection portion.

According to one aspect of the present disclosure, the pair of cylinders may be configured to generate the negative pressure in an alternate manner.

According to one aspect of the present disclosure, the body may be formed of aluminum and has a thickness of 10 mm or more.

According to one aspect of the present disclosure, there is provided a medical suction device includes: the above-described medical suction pump; a suction part connected to the suction pump and including a storage in which objects to be suctioned is stored; and a controller electrically connected to the motor of the medical suction pump and configured to control an operation of the motor.

Further, the medical suction pump and the medical suction device according to the present disclosure may further include other additional configurations without departing from the technical sprit of the present disclosure.

According to one embodiment of the present disclosure, by providing a motor-operated suction pump instead of a hydraulic pump in the related art that causes a relatively large noise of 70 decibel (dB) or more, it is possible to reduce noise during the operation of the suction pump to 45 dB or less and prevent noise stress from being applied to an operator and a patient.

In addition, the size and weight may be reduced as compared with a conventional hydraulic pump to improve mobility of the medical suction device.

### BRIEF DESCRIPTION OF THE DRAWINGS

FIG. 1 is a perspective view of a medical suction device according to one embodiment of the present disclosure.

FIG. 2 is a view schematically illustrating a configuration of the medical suction device according to one embodiment of the present disclosure.

FIG. 3 is a perspective view of a medical suction pump according to one embodiment of the present disclosure.

FIG. 4 is an exploded perspective view of the medical suction pump according to one embodiment of the present disclosure.

FIG. 5 is a partial cross-sectional view of a cylinder and a piston of the medical suction pump according to one embodiment of the present disclosure.

FIG. 6A is a partial cross-sectional view illustrating a linear reciprocating motion of a piston head with a rotation of a crank in the medical suction pump according to one embodiment of the present disclosure.

FIG. 6B is a partial cross-sectional view illustrating a linear reciprocating motion of a piston head with a rotation of a crank in the medical suction pump according to one embodiment of the present disclosure.

FIG. 7 is a plan view of the medical suction pump according to one embodiment of the present disclosure.

### DETAILED DESCRIPTION

Hereinafter, example embodiments of the present disclosure will be clearly described in detail at such an extent that they may be readily practiced by those ordinary skilled in the art.

Throughout the present specification, when a constituent element is referred to as being “positioned”, “located”, or “connected” to one side of another element, the element may be in direct contact with or directly connected to the one side of another element, or may be positioned, located, or connected to another element by intervening yet another element therebetween.

In order to clearly describe the present disclosure, detailed descriptions irrelevant to the present disclosure will be omitted, and the same reference numerals will be given to the same elements throughout the specification. Further, a size, thickness, position and the like of each element illustrated in the drawings are arbitrarily illustrated for the sake of convenience in description, and hence the present disclosure is not necessarily limited to those illustrated. That is, it is to be understood that specific shapes, structures, and characteristics described herein may be modified from one embodiment to another embodiment without departing from the spirit and scope of the present disclosure. Positions or arrangements of individual elements may also be modified without departing from the spirit and scope of the present disclosure.

Therefore, the detailed description described below is not to be taken in a limiting sense, and the scope of the present disclosure is to be taken as covering the scope claimed by the appended claims and their equivalents.

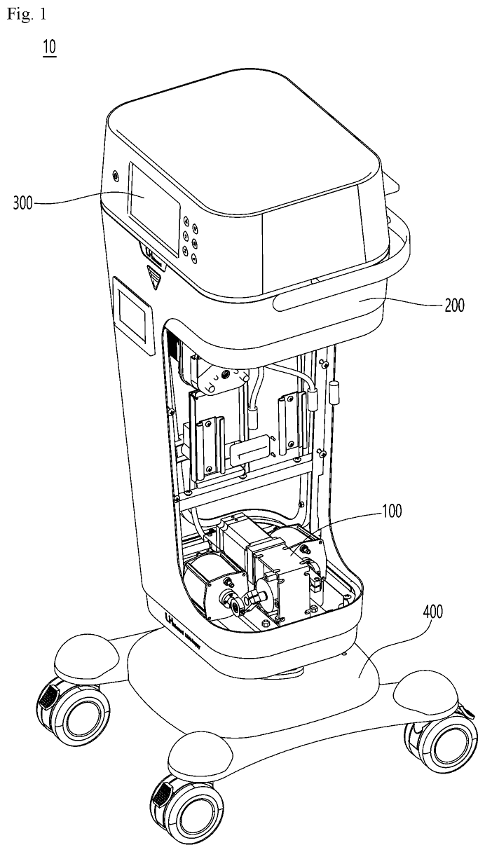

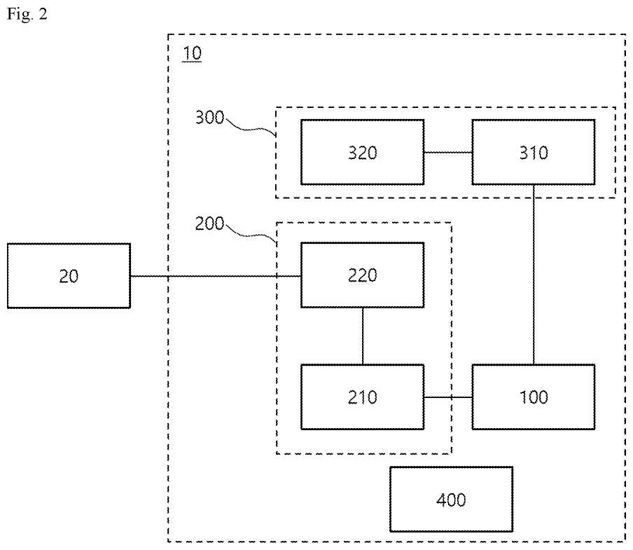

FIG. 1 is a perspective view of a medical suction device 10 according to one embodiment of the present disclosure, and FIG. 2 is a view schematically illustrating a configuration of the medical suction device 10 according to one embodiment of the present disclosure.

Referring to FIGS. 1 and 2, the medical suction device 10 according to one embodiment of the present disclosure may include a suction pump 100, a suction part 200, a manipulation part 300, and a placement part 400.

As shown in FIG. 2, the suction part 200 of the medical suction device 10 according to one embodiment of the present disclosure may include a connector 210 and a storage 220. The connector 210 and the storage 220 of the suction part 200 are not illustrated in FIG. 1.

The medical suction device 10 according to one embodiment of the present disclosure may be connected to, via a tube, a cannula 20 for suctioning objects to be suctioned. When a tissue to be suctioned is crushed and emulsified, or when the cannula 20 is inserted into a body after the tissue is crushed and emulsified, the medical suction device 10 generates a negative pressure in the cannula 20 and the tube connected to the cannula 20 so as to suction the emulsified objects. More specifically, the storage 220 of the suction part 200 may be connected to the cannula 20 via a suction port formed at one end of the storage 220, and may be connected to the suction pump 100 via an exhaust port formed at the other end of the storage 220 and the connector 210 connected to the exhaust port. When the negative pressure is generated in the connector 210 by the suction pump 100, air inside the storage 220 may be discharged via the exhaust port. Thus, an internal pressure of the storage 220 is reduced so that the objects to be suctioned may be introduced into and stored in the storage 220 via the suction port.

The suction pump 100 of the medical suction device 10 according to one embodiment of the present disclosure may be connected to the storage 220 via the connector 210. At this state, the negative pressure is generated by the suction pump 100 so that the objects to be suctioned may be introduced into the storage 220. The operation of the suction pump 100 will be further described later.

The manipulation part 300 according to one embodiment of the present disclosure may include a display 320 and a button section as elements for allowing the user to control the operation of the suction pump 100. As an example, the display 320 may be configured with a touch panel. A user may control the operation of the suction pump 100 by touching a user interface (UI) that appears on the display 320. However, the present disclosure is not limited thereto and manipulation means other than the display 320 and the button section may be used.

Further, the manipulation part 300 according to one embodiment of the present disclosure may include a controller 310 that controls the operation of the suction pump 100 according to a manipulation performed by the user. The controller 310 may be electrically connected to a motor of the suction pump 100 to control an operation of the motor.

The placement part 400 of the medical suction device 10 according to one embodiment of the present disclosure is configured to support elements of the medical suction device 10 such as the manipulation part 300, the suction part 200, the suction pump 100 and the like as described above, thereby enhancing an operational stability of the medical suction device 10. In one embodiment, the placement part 400 is located at the bottom of the medical suction device 10. In one embodiment, the placement part 400 may include wheels which come into contact with the ground. This makes it possible to easily move a position of the medical suction device 10.

According to one embodiment, the placement part 400 may include a vibration absorber. The vibration absorber is located between the placement part 400 and the suction pump 100 and is configured to absorb vibration caused by the operation of the suction pump 100. This can reduce noise during the operation of the suction pump 100.

Next, the suction pump 100 according to one embodiment of the present disclosure will be described in detail with reference to drawings.

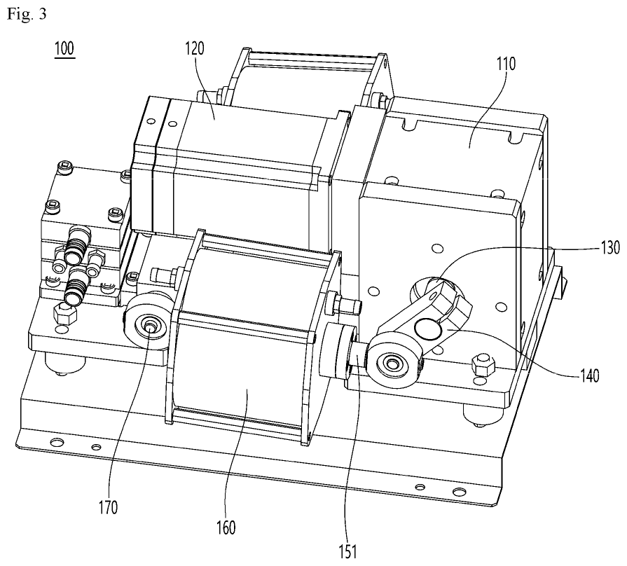

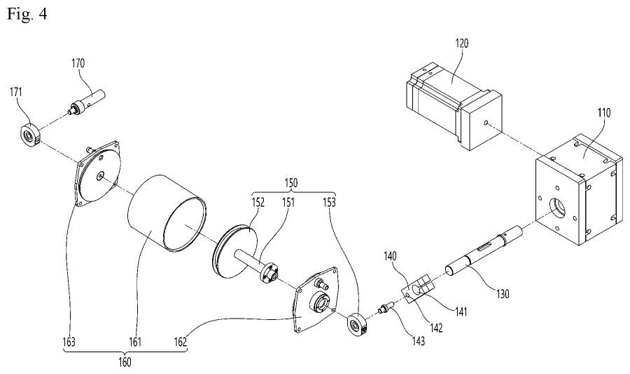

FIG. 3 is a perspective view of the suction pump 100 used in the medical suction device 10 according to one embodiment of the present disclosure, and FIG. 4 is an exploded perspective view of the suction pump 100 used in the medical suction device 10 according to one embodiment of the present disclosure.

Referring to FIGS. 3 and 4, the suction pump 100 according to one embodiment of the present disclosure may include a body 110, a motor 120, a rotational shaft 130, a crank 140, a piston 150, and a cylinder 160.

The body 110 of the suction pump 100 according to one embodiment of the present disclosure is configured to support the motor 120, the rotational shaft 130, and the cylinder 160 so that they may be stably coupled to each other. In FIG. 4, only a portion of the body 110 is illustrated. Parts which fix the motor 120, the rotational shaft 130, and the cylinder 160 correspond to the body 110. A shape of the body 110 is not limited to the form illustrated in FIGS. 3 and 4 but may be changed according to positions, sizes, shapes, and the like of the motor 120, the rotational shaft 130, and the cylinder 160.

According to one embodiment, the body 110 may be formed of a metallic material. For example, the body 110 may be formed of aluminum. The aluminum is a relatively light material metal. By forming the body 110 with the aluminum, it is possible to improve the portability of the medical suction device 10 while maintaining the rigidity thereof. Further, the aluminum is excellent in vibration-absorption and sound-insulation effects. This is effective in improving the operational stability of the medical suction device 10 and suppressing noise. In order to further enhance such a noise suppression effect, the body 110 may have a thickness of 10 mm or more.

The motor 120 of the suction pump 100 according to one embodiment of the present disclosure functions to convert electrical energy into rotational kinetic energy. The motor 120 is connected to the rotational shaft 130 to rotate the rotational shaft 130 in one direction. According to one embodiment, the motor 120 may be connected to the rotational shaft 130 through gears. Based on a gear ratio of the gears, a rotational speed of the rotational shaft 130 with respect to a rotational speed of the motor 120 may be adjusted.

Referring to FIG. 3, the motor 120 may be configured such that a surface of the motor 120 in a direction in which a motor shaft is oriented, is brought into close contact with one surface of the body 110, and the motor shaft penetrates through a wall surface of the body 110 and is inserted into the body 110. Further, the rotational shaft 130 may be configured to penetrate through another surface of the body 110 from the inner side to the outer side. Thus, the gears may be positioned inside the body 110.

The crank 140 of the suction pump 100 according to one embodiment of the present disclosure is configured to convert the rotational motion of the rotational shaft 130 into a linear reciprocating motion. Specifically, the crank 140 may be configured such that one end 141 of the crank 140 is connected to the rotational shaft 130, the other end 142 thereof is connected to the piston 150 via a crank shaft 143. This causes a piston head 152 to reciprocate linearly inside the cylinder 160.

The piston 150 of the suction pump 100 according to one embodiment of the present disclosure includes a connecting rod 151 and a piston head 152. According to one embodiment, the connecting rod 151 may have a rod shape. One end of the connecting rod 151 is coupled to the piston head 152 and the other end thereof is connected to the crank shaft 143. The piston head 152 may reciprocate linearly inside the cylinder 160 as the connecting rod 151 moves by the crank shaft 143. The piston head 152 is configured to push air outward from the interior of the cylinder 160 and may have a plate shape of a predetermined thickness.

In one embodiment, the connecting rod 151 may be connected to the crank shaft 143 via a first bearing 153. The first bearing 153 may reduce friction between the connecting rod 151 and the crank shaft 143 to improve the lifetime of the suction pump 100 and reduce the noise during the operation of the suction pump 100.

The cylinder 160 of the suction pump 100 according to one embodiment of the present disclosure is configured to perform suction and exhaust operations with the linear reciprocating motion of the piston head 152. In one embodiment, the cylinder 160 may include a tubular portion 161 having a constant vertical cross-section, a first sealing portion 162 which seals one end of the tubular portion 161, and a second sealing portion 163 which seals the other end of the tubular portion 161. In one embodiment, the connecting rod 151 of the piston 150 penetrates the first sealing portion 162 and is connected to the crank shaft 143. In this case, the piston head 152 of the piston 150 has a shape corresponding to an inner cross-sectional shape of the tubular portion 161.

The crank shaft 143 circulates at a radius corresponding to a length of the crank 140 with the rotational motion of the crank 140. Thus, when the cylinder 160 is completely fixed to the body 110 and does not move, the linear reciprocating motion of the piston head 152 is limited. To cope with this situation, the cylinder 160 is configured to make a reciprocating angular motion within a predetermined angle through an auxiliary shaft 170 fixed to the body 110, which will be described in detail later.

In one embodiment, the cylinder 160 may be connected to the auxiliary shaft 170 via a second bearing 171. The second bearing 171 may reduce friction between the cylinder 160 and the auxiliary shaft 170 to improve the lifetime of the suction pump 100 and reduce noise during the operation of the suction pump 100.

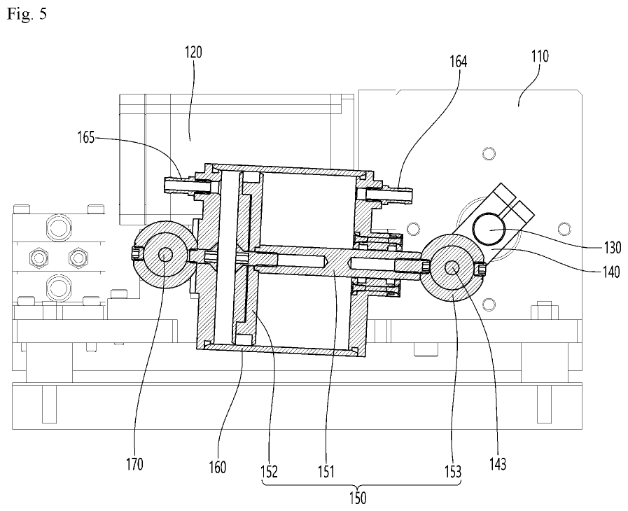

FIG. 5 is a partial side view illustrating cross-sections of the cylinder 160 and the piston 150 of the suction pump 100 according to one embodiment of the present disclosure.

Referring to FIG. 5, suction/exhaust ports 164 and 165 are formed in the cylinder 160. Specifically, each of the suction/exhaust ports 164 and 165 is formed in an internal space of the cylinder 160 which is partitioned into two internal spaces with reference to the piston head 152. Air may be introduced into or discharged from each of the two internal spaces of the cylinder 160 according to the movement of the piston head 152.

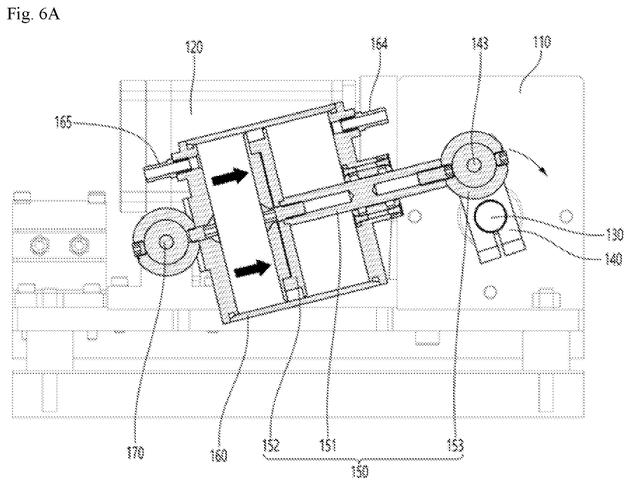

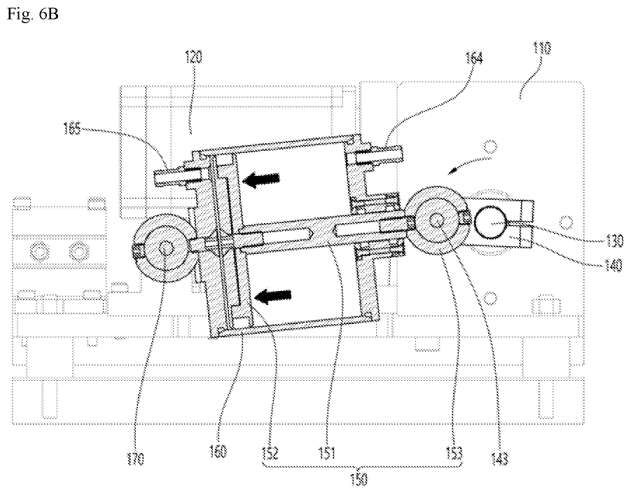

FIGS. 6A and 6B are partial cross-sectional views illustrating the linear reciprocating motion of the piston head 152 with the rotation of the crank 140 of the suction pump 100 according to one embodiment of the present disclosure. FIG. 6A illustrates a state in which the piston head 152 is moved in a direction toward the crank 140 (hereinafter referred to as a “first direction”) inside the cylinder 160, and FIG. 6B illustrates a state in which the piston head 152 is moved in a second direction opposite to the first direction.

Referring to FIG. 6A, as the piston head 152 moves in the first direction, a first internal space defined in the first direction out of the two internal spaces of the cylinder 160, which are partitioned by the piston head 152, becomes narrower. In this case, air is discharged from the first internal space via the suction/exhaust port 164 formed at the side of the crank 140. Meanwhile, as the piston head 152 moves in the first direction, a second internal space defined in the second direction out of the two internal spaces of the cylinder 160 becomes wider. In this case, the negative pressure is generated so that air is introduced into the second internal space via the suction/exhaust port 165 defined in the second direction.

Referring to FIG. 6B, as the piston head 152 moves in the second direction, the first internal space defined in the first direction out of the two internal spaces of the cylinder 160 becomes wider. In this case, the negative pressure is generated so that air is introduced into the first internal space via the suction/exhaust port 164 defined in the first direction. Meanwhile, the second internal space defined in the second direction out of the two internal spaces of the cylinder 160 becomes narrower. In this case, air is discharged from the second internal space via the suction/exhaust port 165 defined in the second direction.

As described above, the suction and exhaust operations are performed along with the linear reciprocating motion of the piston head 152 via each of the suction/exhaust ports 164 and 165. In this case, the negative pressure generated when the air is suctioned into the cylinder 160 is used. The connector 210 of the suction part 200 may be connected to the suction/exhaust ports 164 and 165 to discharge the air in the exhaust operation. Specifically, the suction/exhaust ports 164 and 165 formed at both ends of the cylinder 160 are connected to the connector 210 of the suction part 200. In this case, an exhaust valve may be provided at the end of the cylinder 160 where each of the suction/exhaust ports 164 and 165 is connected to the connector 210, so that the connector 210 may perform only the exhaust operation with respect to the suction pump 100. When the air is introduced from the suction pump 100, the air may be discharged outward via an outlet connected additionally to the exhaust valve. That is, when the air is introduced into the connector 210 from the suction pump 100, the introduction of the air into the connector 210 is blocked by the exhaust valve, and the air is discharged outward via the outlet. When the negative pressure is generated by the suction pump 100, the air is introduced into the suction pump 100 via the exhaust valve. In this way, the connector 210 may perform only the suction operation with respect to the storage 220.

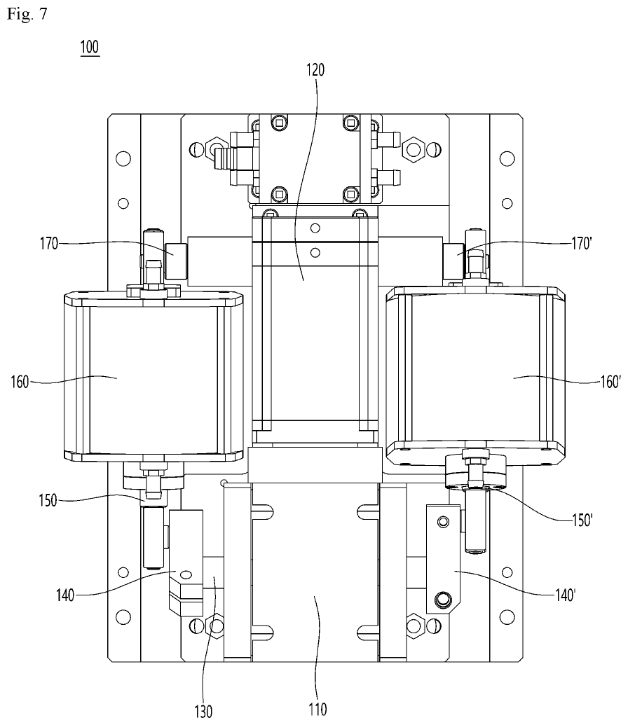

FIG. 7 is a plan view of the suction pump 100 according to one embodiment of the present disclosure.

Referring to FIG. 7, a pair of cylinders 160 and 160′, a pair of pistons 150 and 150′ and a pair of cranks 140 and 140′ may be respectively provided on both sides of the body 110. That is, the pair of cranks 140 and 140′ is respectively connected to both ends of the rotational shaft 130. Each of the pair of cranks 140 and 140′ converts the rotational motion of the rotational shaft 130 into the linear reciprocating motion of each of the pair of pistons 150 and 150′. Thus, the pair of cylinders 160 and 160′ may generate the negative pressure in a simultaneous manner.

According to one embodiment of the present disclosure, one ends of the pair of cranks 140 and 140′ are provided to face each other. Thus, the pair of pistons 150 and 150′ make the linear reciprocating motion at different phases. For example, when the suction/exhaust ports 164 and 165, which are formed in the cylinder 160 provided on the left side among the pair of cylinders 160 and 160′ illustrated in FIG. 7, respectively perform the suction and exhaust operations, suction/exhaust ports 164′ and 165′, which are formed in the cylinder 160′ provided on the right side, respectively perform the exhaust and suction operations. In this way, the pair of cylinders 160 and 160′ may be configured to generate the negative pressure in an alternate manner.

In one embodiment, the suction/exhaust ports 164, 165, 164′ and 165′ of the pair of cylinders 160 and 160′ may be connected to the medical suction device via a T-shaped connector such that the pair of cylinders 160 and 160′ may integrally generate the negative pressure for suctioning the objects to be suctioned. Accordingly, angular momenta caused by the pair of the cranks 140 and 140′ are dispersed in different directions, which reduces load applied to the motor 120. This prolongs the lifetime of the suction pump 100. Further, the friction between the rotational shaft 130 and the body 110 is reduced, which makes it possible to reduce the noise during the operation of the suction pump.

Although the present disclosure has been described above in terms of specific items such as detailed elements as well as the limited embodiments and the drawings, they are merely provided to help more general understanding of the present disclosure, and the present disclosure is not limited to the above embodiments. Various modifications and changes could have been realized by those skilled in the art to which the present disclosure pertains from the above description.

Therefore, the spirit of the present disclosure need not to be limited to the above-described embodiments, and in addition to the appended claims to be described below, and all ranges equivalent to or changed from these claims need to be the to belong to the scope and spirit of the present disclosure.

What is claimed is:

1. A medical suction pump comprising:
a body;
a motor provided in the body;
a rotational shaft connected to the motor and configured to rotate according to a driving of the motor;
a crank having one end connected to the rotational shaft and configured to convert a rotational motion of the rotational shaft into a linear reciprocating motion;
a piston including a connecting rod connected to the other end of the crank and a piston head connected to the connecting rod; and
a cylinder configured to accommodate the piston head therein and having a suction/exhaust port formed therein,
wherein the suction/exhaust port of the cylinder is configured to be connected to a storage in which objects suctioned via a tube are stored, and
wherein a negative pressure is generated when the piston head accommodated in the cylinder moves in a first direction and a second direction opposite to the first direction.

2. The medical suction pump of claim 1, wherein the cylinder, the piston, and the crank include a pair of cylinders, a pair of pistons and a pair of cranks, respectively, which are provided on both sides of the body.

3. The medical suction pump of claim 2, wherein the pair of cranks are configured such that the one ends connected to the rotational shaft faces each other, and
wherein reciprocating motions of the pair of pistons respectively connected to the pair of cranks have opposite phases.

4. The medical suction pump of claim 2, wherein the suction/exhaust port formed in each of the pair of cylinders is connected to a medical suction device via a T-shaped connection portion.

5. The medical suction pump of claim 2, wherein the pair of cylinders are configured to generate the negative pressure in an alternate manner.

6. The medical suction pump of claim 1, wherein the body is formed of aluminum and has a thickness of 10 mm or more.

7. A medical suction device comprising:
the medical suction pump according to claim 1;
a suction part connected to the suction pump and including a storage in which objects to be suctioned are stored; and
a controller electrically connected to the motor of the medical suction pump and configured to control an operation of the motor.
